(19) United States
(12) Patent Application Publication
(10) Pub. No.: US 2025/0196633 A1
(43) Pub. Date: Jun. 19, 2025

(54) INTEGRATED SENSOR AND REMOTE APP FOR AUTOMOBILES

(71) Applicant: John Sowell, Cleveland Heights, OH (US)

(72) Inventor: John Sowell, Cleveland Heights, OH (US)

(21) Appl. No.: 18/980,202

(22) Filed: Dec. 13, 2024

Related U.S. Application Data
(60) Provisional application No. 63/610,131, filed on Dec. 14, 2023.

(51) Int. Cl. B60K 28/06; B60W 40/08; G01N 33/497

(52) U.S. Cl. CPC B60K 28/063; B60K 28/066; B60W 40/08; G01N 33/4972; B60W 2040/0836; B60W 2040/0845

(57) ABSTRACT

A communication warning and safety system having a breathalyzer fail safe app for vehicle status control and notification by wireless communication to detect possible impaired drivers, preventing them from starting the vehicle. An integrated automatic control and response activation of preprogrammed contact information for emergency enabled numbers and two-way communication format provides a variety of action responses based on management communication software preprogramming.

This application claims the benefit of U.S. Provisional Application No. 63/610,131, filed on Dec. 14, 2023.

### BACKGROUND OF THE INVENTION

### 1. Technical Field

This invention is directed to ignition interlock devices to detect alcohol content of the operator and communicate to offsite entities.

### 2. Description of Prior Art

A variety of interlocked devices have been developed to prevent operation of a motor vehicle when an operator is impaired generally as to the consumption of alcoholic beverages, see for example U.S. Pat. Nos. 8,640,813, 6,726,636 and U.S. Publication 2019/0126751.

U.S. Pat. No. 8,640,813 claims an ignition interlock with rolling reset and motion detection which combines a breathalyzer test and motion detection with a reset algorithm for starting the vehicle.

U.S. Pat. No. 6,726,636 is directed to a breathalyzer with voice recognition in a vehicle using a comparison of signals with a speech template and toxic breath setting threshold to determine safe activation of the vehicle.

U.S. Publication 2019/01267151 discloses a built-in breathalyzer for a vehicle which is hard wired to the vehicle's computer to manage computer start actions output commands depending on input from the breathalyzer.

### SUMMARY OF THE INVENTION

A computer smart phone app that communicates with independent or integrated sensors available in new vehicles with the vehicle starting system. The sensing and communication system so enabled will provide vehicle operational status and prevent the vehicle from starting after alcohol level detection enable a contact communication protocol with preprogrammed steps including emergency numbers and sequence depending on status.

### DESCRIPTION OF THE DRAWINGS

FIG. 1 is a graphic flow diagram of the integrated control and contact impairment sensor for automobiles.

### DETAILED DESCRIPTION OF THE INVENTION

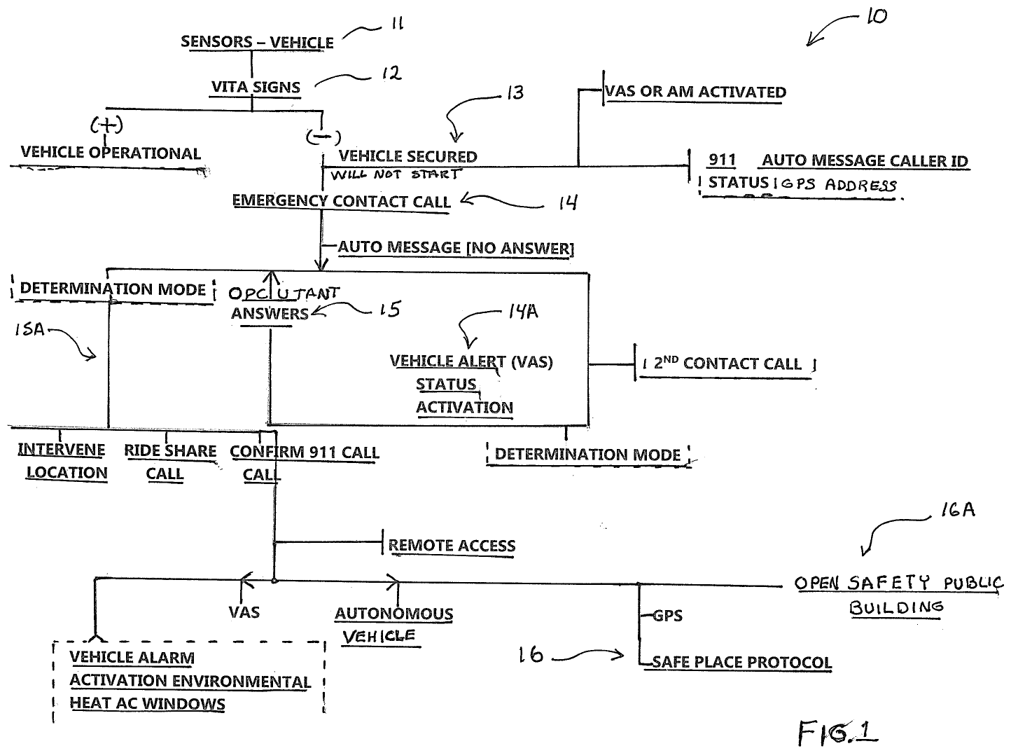

Referring to FIG. 1 of the drawings, the integrated sensor safety and communication application system 10 of the invention can be seen illustrating the activation and action flow diagram enabling the invention to provide an inclusive universal control and emergency contact for the vehicle or like with detection of an impaired or endangered operator dependent on independent input requirements. The system utilizes a variety of sensors to determine alcohol as well as vital signs for assessment and action initiation.

The key aspect of the system 10 of the invention is that it not only disables the vehicle for impaired driver use but provides an interactive communication protocol dependent on the sensor 11 input and vital signs 12 of the driver. When then vehicle is disabled at 13 an emergency contact number is called at 14 which enables vehicle alert, VAS status for communication. If the occupant answers 15 an action determination sequence is initiated at 14 with remote access at 14A is enabled if available on the automobile or VAS equipped vehicle alert systems activating vehicle alarm, for example, lights, etc. with a variety of actions may then be taken based on environmental conditions if required.

Based on sensor determination of temperature activation of AC or heat in the vehicle and if required the windows may be lowered to ventilate.

If an autonomous vehicle is detected by the system, the vehicle can be directed to drive independently to the nearest safe place dependent on predetermined access protocols illustrated at 15, such as a hospital, fire station, EMT, police and/or a secondary open safety public building at 15A.

The operational sequence is as follows. Upon first activation of the system triggered by the breathalyzer detection or sensor detection, the preprogrammed contact number is activated at 14 or if not available default to emergency contact number by a WIFI satellite or cell communication.

Once contact at 15 is made, a prerecorded message may be activated, if required, with the ID of the caller, location of the vehicle and other vital information as well as the caller's physical vitals if so equipped thus monitoring in real-time condition of the operator.

It will be evident that the system of the invention so described can be employed by a variety of user activated apparatus inclusive of private, public and commercial operators, for example. Interlinking communication is therefore enabled by a number of activation equipment format such as vehicle key fob, I-phone app, iPod, or other hand-held remote contact device.

In new vehicle applications the breathalyzer sensor may be mounted in the headliner, steering wheel so that detection of alcoholic content via breath can be achieved. Pre-activation alcohol content level can be predetermined depending on individual circumstances.

It will be apparent that for safety vehicle equipment enablement the engine will be allowed to start in that application thus temporarily overriding the breathalyzer lock out system. It will also be apparent that the application of sensor activation may utilizer AI artificial intelligence systems to enhance usability and autonomous control within the operational application range.

Remote system activation may be needed in certain circumstances such as court ordered programs or other enforceable restrictions imposed on the driver.

Communication enablement would provide, as noted, phone contact access to the GPS in the vehicle for determination of location as is well known within the art.

The application contact of “call ride share” is implicit of commercially available services such as Uber, and Lyft having preprogrammed user identification and address with emergency contact determination by the system.

It will be evident from the above description that a variety of additional elements may be employed as to newer technology, different control parameters and interlinking applications and sensors utilized in an alternate environment to be determined such as the user's clothing.

The issue of EMS access upon arrival will dictate and if needed unlock the vehicle using available interlinked vehicle emergency systems as OnStar, for example. Vehicle cameras may also be employed and accessed to transmit images of the interior, exterior and panoramic surroundings to aid in the safety worker's response.

Other aspects of the system utilization will be apparent to those skilled in the art such as individuals that fall ill or incapacitated or children in the vehicle left attended or unattended via motor and heat sensors activation alarms both on site and remotely using established communication protocols as hereinbefore described.

It will be apparent to those skilled in the art that various changes and modifications may be made therein without departing from the spirit of the invention.

Therefore, I claim:

1. An integrated vehicle sensor safety and autonomous communications system computer application permitting an individual to operate the vehicle comprising,
operational sensors to determine alcohol and medical conditions impairment of an individual,
system activation upon sensor determination of individual's impairment, disabling the vehicle and initiating a communication notification protocol including but not limited to auto emergency contact numbers, 911 call and vehicle alert status (VAS) if equipped.

2. The integrated vehicle sensor safety and autonomous communication system set forth in claim 1 wherein said vehicle sensors comprising,
breathalyzer (alcohol), and individual's vital signs associated with operational impairment by chemical or environmental inducement.

3. The integrated vehicle sensor safety and autonomous communication system set forth in claim 1 wherein said communication notification protocol comprises,
occupant's failure to respond to emergency control number activation response, a series of action determinations enable pre-programmed in the system application.

4. The integrated vehicle sensor safety and autonomous communication system set forth in claim 3 wherein the action determined comprises,
autonomous operation of vehicle, heating, AC control and vehicle's window,
autonomous vehicle activation to direct said vehicle to a safe place dependent on pre-determined access protocols if so equipped.

5. The integrated vehicle sensor safety and autonomous communication system set forth in claim 1 wherein said communication notification protocols further comprise,
occupant answers to auto initiated emergency call response, initiation of a determination mode to access status of individual condition and response required dependent thereon.

6. A method of vehicle operator impairment determination in vehicle safety control operation systems by a computer application comprising the steps of;
a. providing integrated operational condition sensors linked to vehicle's starting and safety systems,
b. activating of communication control protocol including emergency numbers and auto pre-recorded messages,
c. determination of actions based on response of operator thereto,
d. activation of vehicle operational equipment, alarms and remote control via VAS equipped vehicles,
e. directing autonomous operation of vehicle to pre-determined destination based on vehicle location and status,
f. determination of actions including contacting ride share services in response to sequence of operator's status.
